(19) United States
(12) Patent Application Publication
(10) Pub. No.: US 2003/0097178 A1
(43) Pub. Date: May 22, 2003

(54) Length-adjustable ossicular prosthesis

(76) Inventors: Joseph Roberson, Palo Alto, CA (US); John H. Shadduck, Tiburon, CA (US)

Correspondence Address: Joseph Roberson, M.D., California Ear Institute at Stanford, 801 Welch Road, Palo Alto, CA, 94304, US

(21) Appl. No.: 10/262,725

(22) Filed: Oct. 2, 2002

Related U.S. Application Data
(60) Provisional application No. 60/327,093, filed on Oct. 4, 2001.

(51) Int. Cl. A61F 2/18

(52) U.S. Cl. 623/10

(57) ABSTRACT

A middle ear prosthesis that is length-adjustable in a post-implantation period to accommodate tensions in tissue caused by healing. The ossicular prosthesis can be used in total and partial ossicular replacement surgeries and can take the form of (i) an active prosthesis, (ii) a partially-active prosthesis, or (iii) a passive prosthesis. In a preferred embodiment, the prosthesis is partially-active and comprises first and second body portions that slidably mate to alter the overall prosthesis length. An engagement or locking mechanism is provided to lock the first and second body portions in a selected position to thereby provide a selected length. In another embodiment, the prosthesis carries a photothermal micropump for moving fluid between first and second chambers to adjust the prosthesis length. In another embodiment, the prosthesis has a medial portion of a shape memory polymer (SMP) that changes length in response to thermal effects, with the SMP covered with an effective insulator layer to prevent substantial thermal effects in the middle ear.

### CROSS-REFERENCE TO RELATED APPLICATION

This application claims benefit of Provisional U.S. Patent Application Ser. No. 60/327,093 (Docket No. JHR-001) filed Oct. 4, 2001.

### BACKGROUND OF THE INVENTION

1. Field of the Invention

The present invention relates to an ossicular prosthesis, and more particularly to a middle ear prosthesis that is length-adjustable in a post-implantation period to accommodate tensions in tissue caused by healing.

2. Description of the Related Art

The middle ear comprises a levered vibrating system for sound transmission from the tympanic membrane (eardrum) to the inner ear. The air-filled volume of the inner ear contains three middle ear bones or auditory ossicles: the malleus 2, the incus 4 and the stapes 6 (see FIG. 1). The malleus has a handle portion that contacts the tympanic membrane 7 and a head portion that couple with the incus. The stapes includes an arch, formed by a pair of limbs, and a footplate. The footplate communicates with the oval window 8 that leads to the inner ear. As the malleus handle vibrates in response to sound waves striking the tympanic membrane, the head portion of the malleus couples the vibrations to the incus, and thereafter to the arch of the stapes. The stapes footplate in turn couples the auditory vibrations to the inner ear. The shape and structure of the ossicles create a lever action within the middle ear to amplify vibrations. Thus, a greater vibrational force is generated at the oval window 8 than at the tympanic membrane.

An ossicular prosthesis is an implantable medical device that is adapted to replace one or more ossicles when the middle ear structures are damaged. At present, there are number of commercially available ossicular prostheses, in general, for total or partial reconstruction of the middle ear. A total prosthesis is designed to replace the malleus, incus and superstructure of the stapes. A partial prosthesis is designed to replace the malleus and the incus only.

In order to function as vibration coupling system, an ossicular prosthesis must have a precise length dimension to fit each patient's anatomy to thereby transmit sound from the tympanic membrane to the inner ear. As currently practiced, a reconstructive surgery of the ossicles utilizes a fixed length prosthesis that can have various shapes, sizes and configurations. It has been found that tissue healing following surgery can produce small alterations in the physical dimensions of the native middle ear tissues and/or the healed tissues about the site of the incision (typically around the eardrum). Frequently, an implanted prosthesis will have an inappropriate overall length following the healing process. FIGS. 2A-2B provide a graphic illustration of one type the problem that can occur following surgery, wherein FIG. 2A illustrates a fixed length prosthesis shortly after implantation. The proximal end of the prosthesis is rapidly secured to the eardrum by epithelial growth. The distal end of the prosthesis (not shown) is similarly secured to another middle ear structure. FIG. 2B depicts the eardrum after healing when tensions can cause flattening of the eardrum or movement slightly in the direction of the arrows. Since the prosthesis has a fixed length, the tissue tensions can pull the eardrum slightly away from the prosthesis and leave a space or other less secure engagement. While vibration coupling can still function well at some frequencies, other frequencies (e.g., low Hz vibrations) may not transmit at all through the prosthesis.

In other less frequent cases, the physical dimensions of a patient's middle ear will change over time due to aging and other factors-thus causing deterioration of the performance of the prosthesis. What is needed is a new system for ossicular prostheses that allows for post-implantation length adjustment to overcome variations in healing.

### SUMMARY OF THE INVENTION

In general, the apparatus of the present invention provides a length-adjustable prosthesis that allows post-implantation “tuning” of the prosthesis after healing has occurred and physical tissue dimensions have become static. Such a post-operative adjustment of the prosthesis length will allow for resumption of proper hearing function. The invention will prevent the need for re-operation. The deterioration of hearing function can thus be avoided. The apparatus is adapted for total ossicular reconstruction or partial ossicular reconstruction (e.g., a malleus-head prosthesis, malleus-footplate prosthesis; stapes-bucket prosthesis; stapes-piston prosthesis).

More in particular, the present invention provides a middle ear prosthesis that is length-adjustable in several different embodiments for different total and partial ossicular replacement surgeries. The prosthesis can be active, partially active or passive. In one preferred embodiment, the prosthesis is partially-active and comprises first and second body portions that slidably mate, for example, in the form of shaft of one body that slides in a bore in the other body. An engagement or locking mechanism is provided to lock the first and second body portions in a selected position to thereby provide a selected length. The locking mechanism comprises a piezoelectric or nickel titanium collar in the bore of one element that grips or engages the shaft of the other body element in a first condition. The invention further provides an external energy source that can develop voltage or a thermal effect in the prosthesis to cause the collar to disengage thereby allowing the prosthesis to adjust to a different length in response to tensions in the healed tissue.

Several other embodiments are described below that utilize an external energy source to either actively, or partially actively, provide adjustment means to alter the length of the prosthesis.

### BRIEF DESCRIPTION OF THE DRAWINGS

Other objects and advantages of the present invention will be understood by reference to the following detailed description of the invention when considered in combination with the accompanying Figures, in which like reference numerals are used to identify like elements throughout the disclosure.

FIG. 1 is a schematic view of the outer ear, middle ear and inner ear that show the location of the auditory ossicles.

FIG. 2A is a schematic view of a patient's tympanic membrane and its engagement with a prior art prosthesis shortly after reconstructive surgery.

FIG. 2B is a view similar to FIG. 2A showing the relationship of the patient's tympanic membrane and the prior art prosthesis after the healing process has displaced the tympanic membrane away from the head of the prosthesis.

FIG. 3A is a perspective view of an exemplary Type “A” partially-active middle ear prosthesis corresponding to the present invention that defines a selected length between first and second ends thereof.

FIG. 3B is a view of the prosthesis of FIG. 3A after adjustment to another selected length between the first and second ends thereof.

FIG. 4 is partial cut-away view of the prosthesis of FIGS. 3A-3B.

FIG. 5 is block diagram of circuitry of the prosthesis of FIGS. 3A, 3B & 4.

FIG. 6A is a cutaway view of an alternative Type “A” middle ear prosthesis.

FIG. 6B is a cutaway view of an alternative Type “A” middle ear prosthesis that utilizes a nickel titanium alloy locking collar.

FIG. 7 is a cutaway view of an exemplary Type “B” active middle ear prosthesis that utilizes a microfluidics system to adjust the prosthesis to a longer or shorter overall length.

FIG. 8 is a cut-away view of a microfluidics structure of the prosthesis of FIG. 7.

FIG. 9 is a sectional view of an alternative embodiment of a Type “B” prosthesis.

FIG. 10 is a sectional view of an alternative Type “B” prosthesis.

FIG. 11 is a cutaway view of a portion of an exemplary Type “C” passive prosthesis that utilizes a microchannel structure for a graduated fluid transfer based self-adjustment of the prosthesis length.

FIG. 12A is a sectional view of a Type “D” prosthesis fabricated of a shape memory polymer.

FIG. 12B is a sectional view the prosthesis of FIG. 12A with the shape memory polymer being irradiated and thereby being adjusted to another length.

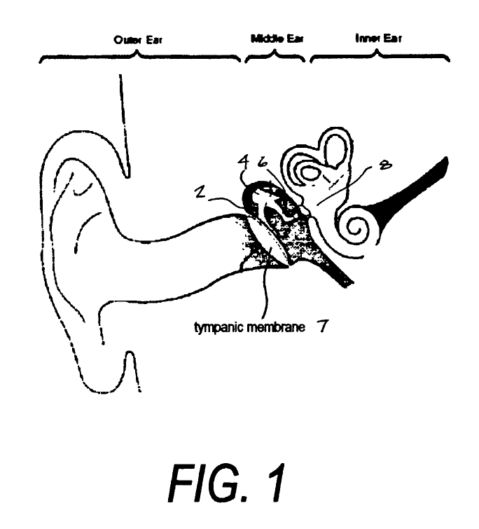

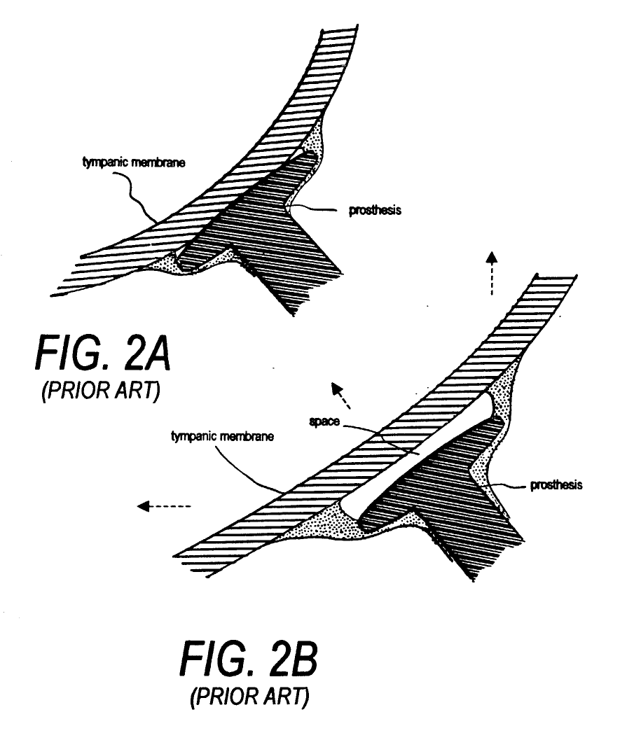

### DETAILED DESCRIPTION OF THE INVENTION

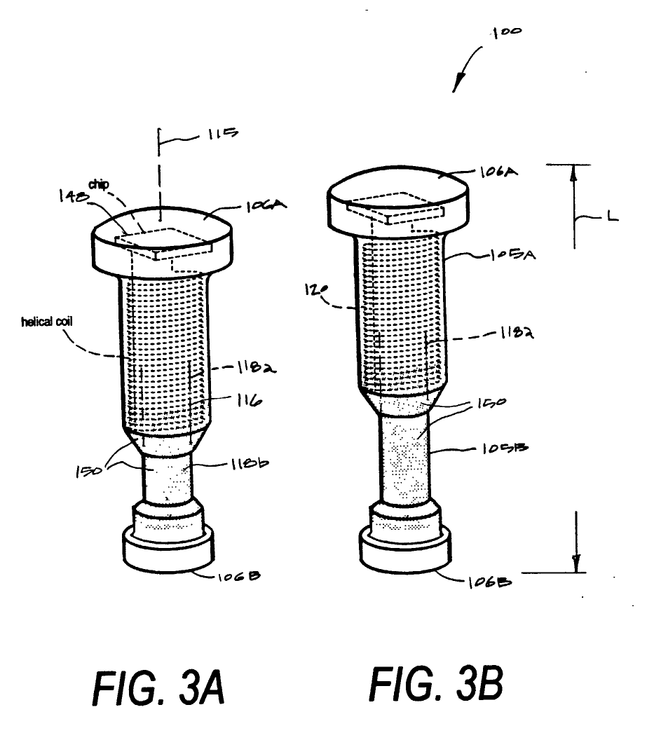

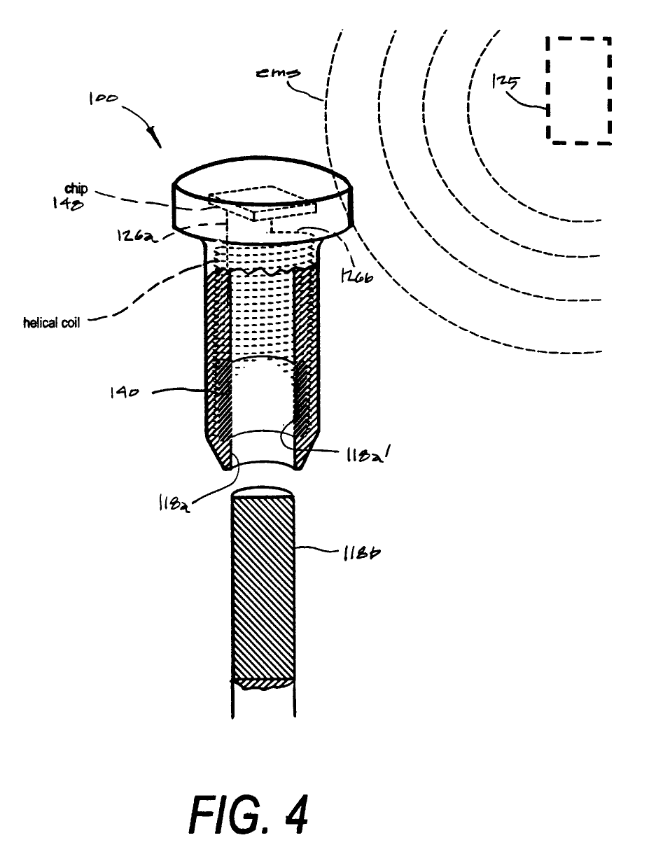

1. Type “A” ossicular prosthesis. An exemplary Type “A” prosthesis 100 corresponding to the invention is illustrated in FIGS. 3A-3B and 4 that is adapted for post-implantation length adjustment. In general, the Type “A” embodiments of the invention are herein called partially-active systems, in that a remote energy source or external input is used to allow adjustment of first and second body portions 105A and 105B relative to one another by means of an engagement and/or disengagement mechanism. Thus, the body portions can be maintained or locked relative to one another to provide a selected overall prosthesis length. In the Type “A” system, the actual adjustment of the prosthesis length is caused in one or more time intervals as a response to tensions applied to the prosthesis by the tissue during the healing process. Types “B” and “C” prosthesis systems will be described seriatim below, and cover (i) active systems that utilize an external input to positively drive the overall length of the prosthesis, and (ii) passive systems that adjust length in response to tensions within tissue without external inputs.

Referring to FIGS. 3A and 3B, the Type “A” prosthesis 100 comprises first and second body portions 105A and 105B that are axially moveable relative to one another. The prosthesis has a proximal (first) or head engagement surface indicated at 106a that is adapted for engaging a structure of the patient's middle ear, for example the tympanic membrane. The prosthesis has a distal (second) foot engagement surface indicated at 106b that is shaped and dimensioned for engaging another middle or inner structure, for example the stapes footplate. It should be appreciated that the head and foot engagement surfaces 106a-106b can have any suitable shape and dimension or engaging middle ear structures in a total or partial ossicular replacement surgery, and the configuration shown in FIGS. 3A-3B is for convenience only. Other elements, such as bails, hoops or leg portions (not shown), may be affixed to an end of the prosthesis as is common for coupling with middle ear structures in a partial middle ear reconstruction.

FIG. 4 shows a partial cut-away view of the prosthesis 100 that extends along axis 115 and defines an overall length dimension indicated at L. The first and second body portions 105A and 105B slidably mate along an interface 116 that in this embodiment comprises the interface between bore 118a in first body portion 105A and the outer surface of shaft portion 118b of second body portion 105B. The bore and shaft portions, 118a and 118b, may have any suitable cross-sectional shape, dimension or any other suitable cooperating arrangement.

Of particular interest, referring to FIG. 4, the first body portion 105A carries therein a helical coil 120 (phantom view) that is tuned to be responsive to electromagnetic frequency transmitted by an external Rf transmitter or source indicated at 125. The helical coil 120 has first and second leads 126a and 126b extending from coil ends to circuitry components, such as a capacitors and diodes, to thereby cause electrical current flow within the implant to actuate an engagement-disengagement mechanism between the cooperating first and second body portions 105A and 105B. The Rf transmitter 125 can be a device that is positionable outside the outer ear of the patient, or it can have a working end that is dimensioned for partial insertion into the patient's car canal.

More in particular, still referring to FIG. 4, the first body portion 105A carries a collar 140 of a piezoelectric material that has a bore portion 118a′ extending therethrough. The outer surface 142 of piezoelectric collar 140 is bonded to first body 105A. The bore portion 118a′ in the piezoelectric collar 140 is adapted to move between a repose contracted diameter d₁ and a second expanded diameter d₂ upon a voltage change in the material. Thus, the piezoelectric material will engage and grip the outer surface of shaft portion 118b of second body 105B until a voltage change can cause transient disengagement thereof. The piezoelectric effect is well known in the art and describes a coupling between a material's mechanical and electrical behaviors. In basic terms, when a piezoelectric material is subjected to a voltage change, it will mechanically deform to a certain extent. Conversely, when a piezoelectric material is compressed, an electric charge collects on its surface. A number of crystalline materials exhibit piezoelectric behavior. On a nanometric scale, the piezoelectric effect results from a non-uniform charge distribution within a crystal's unit cells. When such a crystal is mechanically deformed, the positive and negative charge centers displace by differing amounts. So while the overall crystal remains electrically neutral, the difference in charge center displacements results in an electric polarization within the crystal. Electric polarization due to mechanical input is perceived as piezoelectricity.

The exemplary embodiment of FIG. 4 depicts the piezoelectric element as a collar that can be any suitable axial and radial dimension, within the bore 118a in first body 105A. Alternatively, the first body 105A can be substantially entirely fabricated of a piezoelectric crystal with suitable biocompatible coatings, as required. In the embodiment of FIGS. 3A and 4, the piezoelectric collar “disengages” by expanding the bore 118a′ within the piezo-element. It should be appreciated that the system also can provide a shaft portion the second body 105B that expand/contracts to engage and disengage from the bore of the first body 105A.

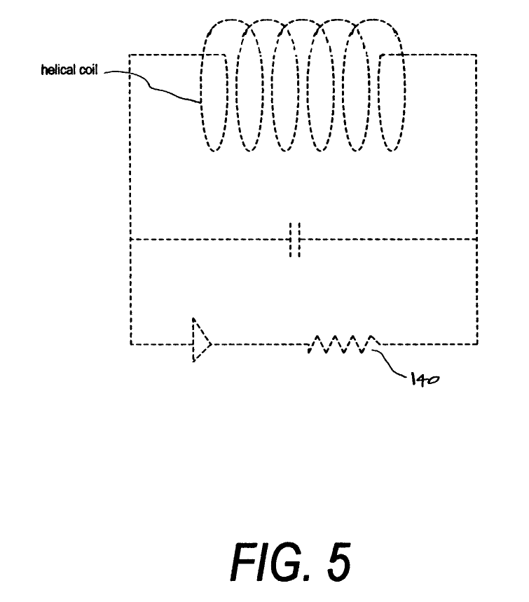

The first and second body portions 105A and 105B of the implant can be fabricated from any suitable biocompatible material, such as hydroxyapatitc or titanium that are known in the fabrication of ossicular prostheses. The collar 140, for example, can be fabricated from lead titanate zirconate ceramics known in the art (e.g., PZT-2, PZT4, PZT4D, PZT-5A, PZT-5H, PZT-5J, PZT-7A or PZT-8). The helical coil 120 can be carried in any part of the first body 105A for electrical coupling to piezoelectric collar 140. For example, the coil 120 carries an insulated coating and can be fabricated into molded polymeric body, or insulatively bonded around an exterior of a machined or cast body. The piezoelectric collar 140 is coupled to by circuitry to coil 120 which can take any number of forms. FIG. 5 shows a block circuitry diagram illustrating an external Rf transmitter source 125 that can propagate an electromagnetic signal ems or wave to the helical element 120 (i.e., coil or antenna) of the prosthesis 100 (see FIG. 4). The electromagnetic signal ems has a selected frequency, amplitude, wave form, power level and repetition rate to cause current flow through the helical coil (and to optional capacitors, power rectifiers, diodes and other circuitry components) to thereby deliver a transient voltage change to the piezoelectric collar 140 to alter the diameter of bore portion 118′ from its contracted diameter d₁ to its expanded diameter d₂. FIGS. 3A and 3B indicate the required circuitry is contained in a “chip” indicated at 148 carried in body 105A that can be any silicon chip or other type assembly that can be produced in sufficiently small dimensions, e.g., as known in the MEMS field.

FIGS. 3A and 3B further depict a surface coating 150 carried only proximate to the mating surface portions of first body 105A and second body 105B that is adapted to reduce epithelial growth over the surfaces for a period of time. While the ends of the prosthesis preferably are rapidly fused to the interfacing anatomic structures by tissue layers forming about the body surfaces, it would be preferred to have less tissue accumulation about the mating interface to allow slidable movement of the body portions about the interface 116 when the disengagement mechanism is actuated. As one example, a biocompatible pyrolytic carbon coating can be used.

In a method of use corresponding the invention, the surgeon would adjust the length of the prosthesis 100 prior to surgery, or during surgery, based on imaging and physical measurements of the optimal prosthesis length. The piezo-element then would assume its repose state with the first and second body portions 105A and 105B “engaged” to provide the selected length L prior to implantation. During the initial 24 to 72 hours post-implantation, the first and second end surfaces 106a and 106b of prosthesis would be subject to at least partial epithelial overgrowth that thereby secure the ends of the prosthesis to the targeted anatomic structures. At one or more points in time following the implant procedure, for example, from about 48 hours to about 4 weeks after the procedure, the system of the invention would be activated to disengage the first and second body portions 105A and 105B by means of the piezoelectric collar 140 to thereby allow adjustment of the length L of the prosthesis. The actual adjustment of the length occurs in response to very slight tensions that build in native tissues as the incisional wounds about the tympanic membrane heal. Since the tissue response may be slow in adjusting the prosthesis to provide an untensioned condition (with respect to native tissues), the scope of the invention includes disengaging the first and second body portions 105A and 105B for any selected time interval (e.g., from 1 s. to 1 hr.), or any sequence of intermittent time intervals, over any number of days or weeks in the post-implantation time period. The scope of the invention further includes use of a pre-programmed, automated Rf transmitter system for use in the patient's home, or the use of the transmitter system in the physician's office following standard tests of the patient's frequency responses.

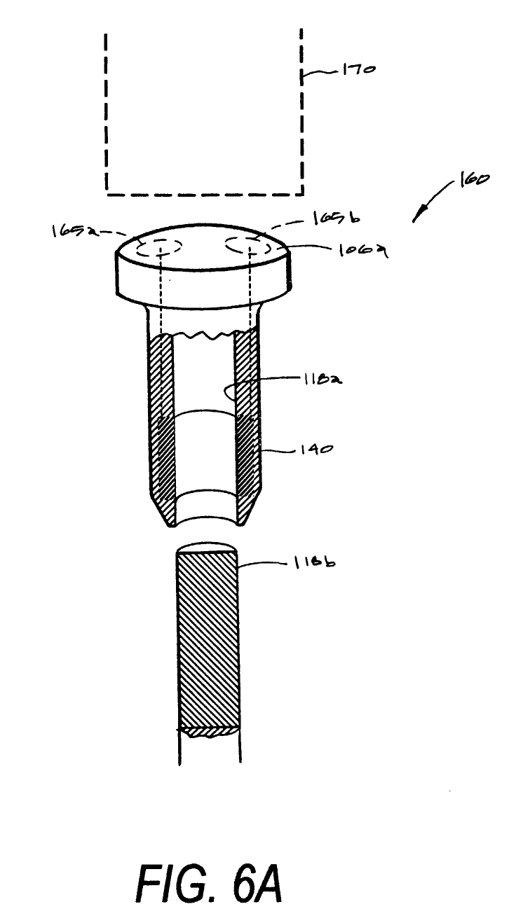

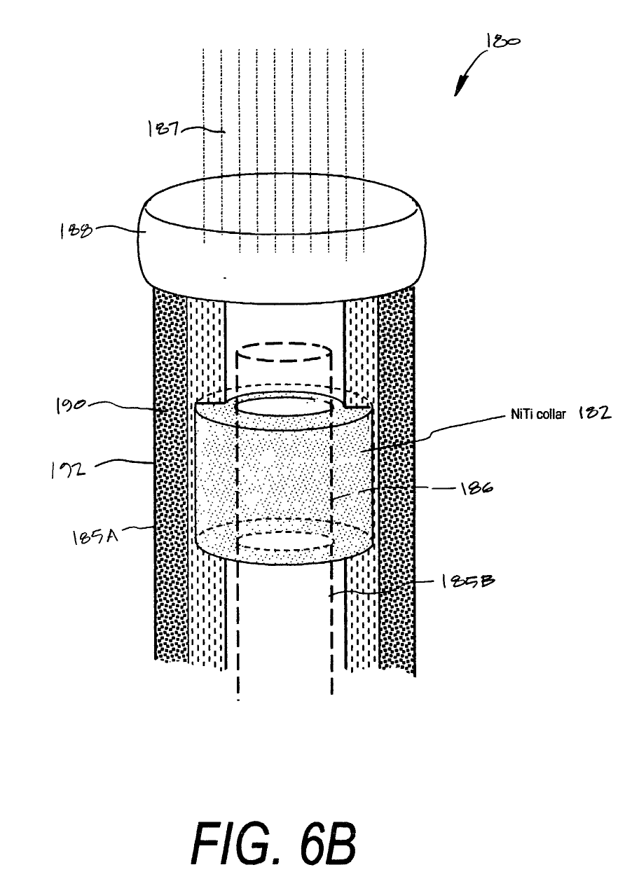

FIG. 6A illustrates another Type “A” prosthesis 160 that functions substantially the same as the previously described embodiment. This embodiment is adapted only for those implants that have a proximal (first) surface 106a that contacts the eardrum, for example in a total ossicular replacement prosthesis. As can be seen in FIG. 6A, the proximal surface 106a has at least one electrode that is exposed (or covered with a very thin electrically transmissive layer). In this embodiment, the prosthesis has two opposing polarity electrodes 165a and 165b in proximal surface 106a that are coupled to circuitry similar to that described previously (see FIG. 5) coupled to the piezoelectric element 140. FIG. 6 shows a “contact” form of emitter 170 that can be placed in contact with the eardrum in opposition to electrodes 165a and 165b to thereby pass low voltage current to the circuitry of the prosthesis. It is believed that a sufficient charge can be built up rapidly to actuate the piezoelectric element, in combination with a capacitor, to allow length adjustment of the prosthesis. In all other respects, the method of the invention compares to that described previously. Thus, the scope of the invention includes any length-adjustable ossicular prosthesis (i) that carries a coil or antenna for receiving an electromagnetic energy transmission from an external “non-contact” source that is convertible to voltage or current within the implant for actuating any length-adjustment mechanism, or (ii) that carries an exposure of at least one electrode for coupling with a “contact” electromagnetic source for similar purposes.

In another embodiment shown in FIG. 6B, the prosthesis 180 is similar to that of FIGS. 3A-6A, except that the collar portion 182 that changes in dimension to release the first and second members 185A and 185B to slide relative to one another is fabricated of a nickel titanium (NiTi) shape memory alloy (SMA) that is designed to alter its dimension upon being elevated to a selected temperature. The NiTi alloy collar 182 can be designed to increase its bore diameter to release its engagement of shaft portion 186 at a selected temperature just above body temperature, e.g., between about 38° C. and 42° C. The NiTi alloy collar 182 can be elevated in temperature by resistive heating when connected to an electrical source and circuitry similar to that shown in FIG. 6A. However, as depicted schematically in FIG. 6B, the NiTi alloy collar 182 can be heated with a radiation 187 from a light source that has a selected wavelength to penetrate the eardrum and a transparent body portion 188 of the prosthesis to elevate the temperature of the collar 182. The light source can be a laser or non-laser source that provides a wavelength in the IR spectrum, for example. The inner ear is sensitive to temperature changes. Therefore, the entire prosthesis is coated with any suitable high performance insulative material indicated at 190. One example of an insulator that may be used in the invention in a thermally conductive graphite foam disclosed in U.S. Pat. Nos. 6,037,032 and 6,033,506. This graphite foam has an open microcellular structure that makes it much lighter than other materials used in thermal management systems, and may be coated with another suitable non-permeable coating 192. It is believed that only very low temperature changes will be required to actuate the NiTi collar, and the scope of the invention includes any type of nickel titanium actuator-type mechanism that have been developed in the prior art. A number of nickel titanium actuator and SMA mechanisms have been developed by TiNi Alloy Company, 1619 Neptune Drive, San Leandro, Calif. 94577.

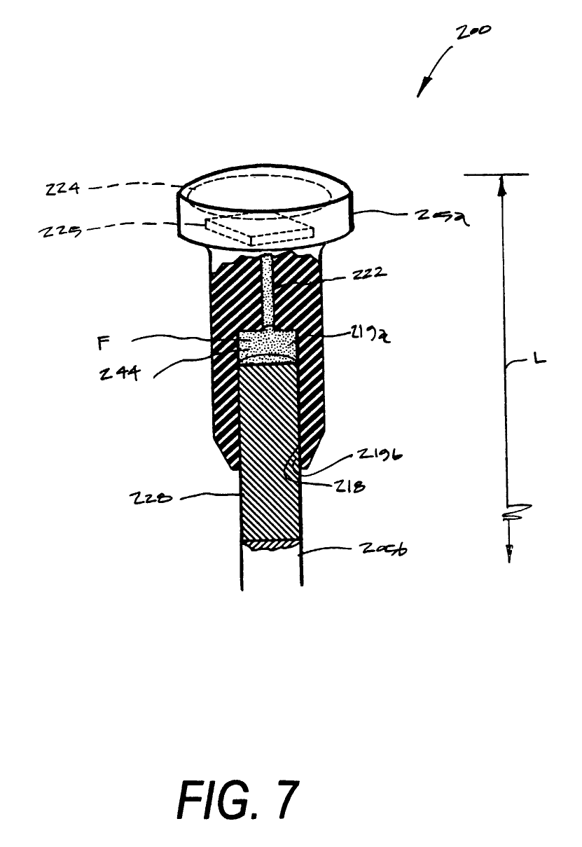

2. Type “B” ossicular prosthesis. An exemplary Type “B” prosthesis 200 corresponding to the invention is illustrated in FIG. 7, which is herein called an active system in that a remote energy source or input is used to positively adjust the first body 205A relative to the second body 205B, which otherwise reciprocate about interface 116 as described previously. In one embodiment, the Type “B” prosthesis again carries a helical coil 120 (not shown) that can be coupled within an external energy emitter 125 to provide voltage/current within the implant. In this embodiment, the basic active implant of FIG. 7 uses an energy source to provide voltage to drive a microfluidic system that uses fluid flows between chambers to adjust the length L of the prosthesis. The use of an electrical charge to cause flows in or through a microchannel structure is known in the art, and one manner of developing electrically-induced flows is described in the following materials which are incorporated herein by this reference: Conlisk et al., Mass Transfer and Flow in Electrically Charged Micro- and Nanochannels, Analytical Chemistry, Vol. 74 Issue 9, pp. 2139-2150; also see the article at http://www.sciencedaily.com/releases/2002/05/020506074547.htm titled Electricity Can Pump Medicine in Implanted Medical Devices.

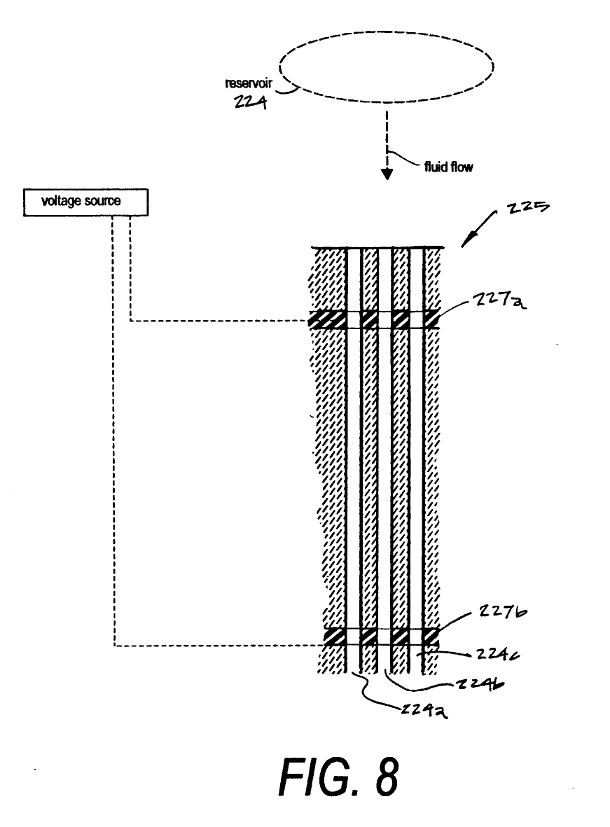

FIG. 7 shows that bore 218 in first body 205A has a proximal end 219a and a distal end 219b. The proximal end 219a interfaces with an end of at least one inflow-outflow channel 222 (collectively) that communicates with a reservoir 224 (phantom view) in a more proximal portion of first body 205A. FIG. 8 illustrates a greatly enlarged view of an array of microchannels 224a-224n that can be fabricated in a silicon chip 225 along with circuitry for causing directional fluid flows within the microchannels. In FIG. 8, the microchannels 224a-224n in the chip assembly define a length dimension d and carry opposing polarity electrodes 227a and 227b at first and second ends of each channel. The electrodes 227a and 227b are connected by leads to a voltage source as described previously (i.e., helical coil and circuitry). FIG. 8 further shows a charge-responsive fluid F carried in reservoir 224 that can be induced by to migrate from reservoir 224 to chamber portion 244 of bore 218 by a charge within the microchannels between the spaced apart electrodes. The migration of fluid F to chamber portion 244 thus can move shaft portion 228 of second body 205B distally by hydraulic forces. The components may be provided with seals, O-rings or the like to make the system substantially fluid-tight. The fluid F can be any suitable biocompatible fluid, such as a concentrated saline solution that is responsive to an electrical charge. By using the above-described mechanism, and reversing the polarity of the electrodes at the channel ends, the length L of the prosthesis can be adjusted the opposite direction. The force of the fluid flows in the just-described system is limited, and therefore the method of use can rely partly on tissue tensions as described previously. In other words, the electrical charge would be applied for any desired time interval-and the combination of tissue tensions developed by the healing process and the fluid flows will move the prosthesis from an initial length to an adjusted length.

In another embodiment (not shown), the voltage source previously described can drive a micropump, impeller, or peristaltic pump arrangement in the microchannels 224a-224n to cause fluid flows if it is found that higher hydraulic forces are useful.

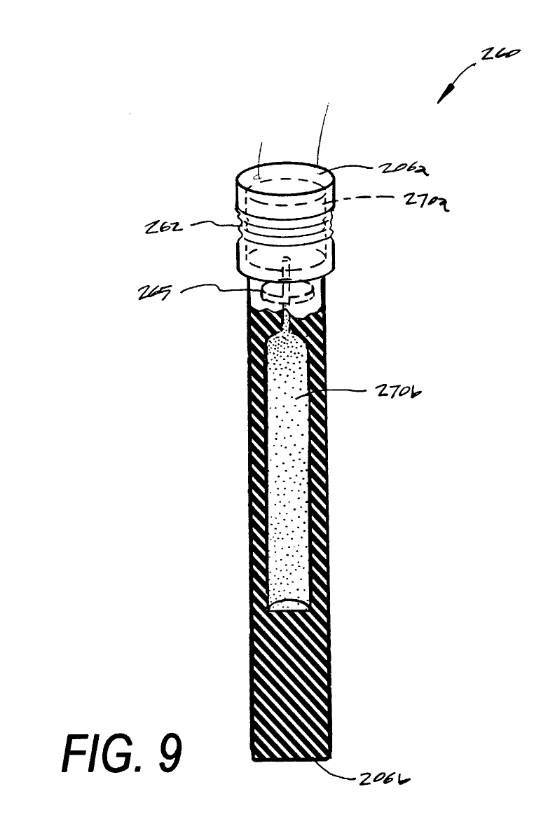

It should be appreciated that the systems described above for moving fluids between first and second chambers can also be used to expand or contract an expandable member such as a balloon or bellows structure to shorten or lengthen the prosthesis. For example, FIG. 9 depicts a prosthesis 260 of a unitary member that has an expandable bellows portion 262 at a proximal portion thereof. The electrical-driven microfluidic system is indicated at 265 which can drive fluid from first chamber 270a to second chamber to 270b to adjust the length of the prosthesis. The prosthesis again carries a helical coil 120 (not shown) therein that is coupled within an external energy emitter. This type of prosthesis would be adapted for use in, for example, partial ossicular replacement surgeries wherein length-adjustment would be minimal. In another embodiment (not shown) the expandable member can be in a medial portion of the prosthesis to swell or contract the transverse section of axially-extending webs of prosthesis to thereby slightly adjust the overall length.

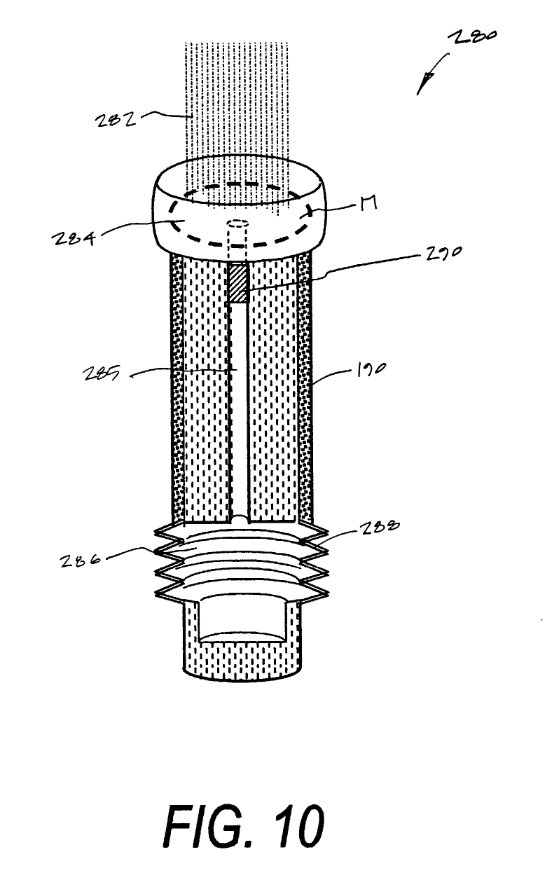

In another embodiment, referring to FIG. 10, a length adjustable prosthesis 280 is shown that is similar to that of FIG. 9 that cooperates with a light source for delivering energy via light beam 282 to an internal chamber 284 that carries a fluid media M such as silicone or water. The light beam 282 can elevate the fluid media temperature slightly to cause its expansion to thereby cause it to flow through channel 285 to expand the fluid volume in the chamber 286 and extend the bellows portion 288. The system further provides a one-way valve 290 as is known in the art to lock the expanded fluid volume in the chamber 286. It can be seen that the prosthesis can be extended, and locked in an extended position, by use of this system which comprised a photo-actuated pump system. It should be appreciated that the scope of the invention extends to any prosthesis that carries a photo-actuated pump system and further extends to photo-actuated valve systems that can be combined with the fluid pump system.

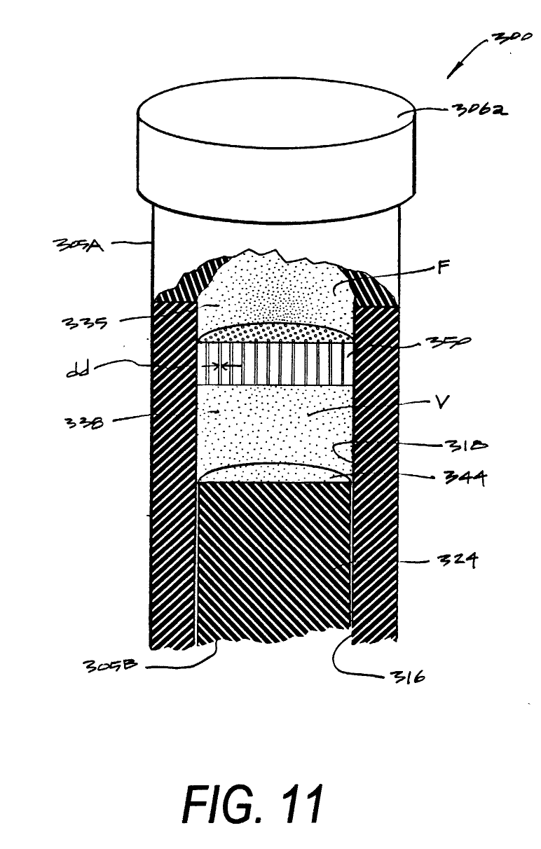

3. Type “C” ossicular prosthesis. An exemplary Type “C” prosthesis 300 corresponding to the invention is illustrated in FIG. 11, which is herein called a passive system in that remote energy sources or inputs are not relied upon to adjust the overall length of the prosthesis. FIG. 11 more particularly shows a cut-away view of the proximal end of prosthesis 300 which comprises first body 305A and second body 305B. The body portions can axially reciprocate along an interface 316 which is again shown as the interface of a bore 318 in the first body 305A cooperating with shaft portion 324 of second body 305B. This system embodiment, however, provides the prosthesis with self-adjusting capabilities.

In FIG. 11, first body 305A has a reservoir or chamber 335 that is proximal to the proximal end 338 of bore 318. The chamber 335 carries a biocompatible fluid F such as sterile water or silicone, as does the proximal end 338 of bore 318 that interfaces with the proximal end 344 of shaft 324. Of particular interest, a microchannel structure 350 is carried in a fixed position by the prosthesis 300 between chamber 335 and the proximal end 338 of bore 318. By the term microchannel structure, it is meant that the structure carries channels, pores or openings that generally define a very small dimension dd for restricting fluid flows therethrough. The dimension dd thus defines the maximum size molecules that can easily flow therethrough.

In the method of use corresponding to the invention, the channel diameter, or combination of diameter and length, is selected to allow fluid F to flow very slowly from chamber 335 to the proximal end 338 of bore 318, and vice versa, in response to axial tensions placed on the prosthesis during the healing process. For example, it is believed that epithelialization of the first and second ends of the prosthesis will occur very rapidly after implantation to thereby secure the ends of the prosthesis to the targeted anatomic structures. Thereafter, surgical would healing may occur over 2 to 4 weeks post-implantation. Any wound contraction or other changes in the native anatomy then can apply very slight axial tensions to the prosthesis to cause fluid F to flow through the microchannel structure 350 and alter the volume V of fluid in the proximal end 338 of bore 318 thus resulting length self-adjustment. (It should be appreciated that chamber 335 is fabricated with a relief valve, bladder, flexible wall portion or other pressure compensation mechanism (not shown) to prevent hydraulic locking of the shaft in the bore).

At the same time, the typical acoustic loading conditions, for example frequencies from 250 to 8,000 Hz, will not cause the flow of fluid F between the chambers due to the selected restrictive dimensions of the channels. Thus, it is believed that the prosthesis will provide acoustic coupling, for example, from the eardrum to the inner ear, even though the fluid F forms part of the transmission chain. The diameter of the channels, or the maximum pore dimension, is somewhat larger than the molecular size of the fluid, and preferably is in the range of about 0.25 μm to 50 μm, and more preferably from about 0.5 μm to 10 μm, e.g., when water is used as fluid F. The microchannel structure 350 can be fabricated by various MEMS techniques or can be a sintered metal that develops a selected pore dimension. The functionality of the microchannel structure 350 and fluid flow therethrough is intended to extend only through the post-implantation time interval that includes the wound healing period. At a certain point in time, epithelial growth will cover the prosthesis to prevent further self-adjustment.

The present invention contemplates other passive implants (not shown) that have mating first and second body portions that are slidable relative to one another to self-adjust during the healing process. Thereafter, an internal portion of the otherwise passive implant can be provided with a bonding agent to bond and lock the first and second body portions in a fixed relationship to provide a selected overall length. For example, in a total replacement prosthesis, a very fine needle can be introduced through the eardrum and through a pierce-able port in the proximal surface of the implant that is otherwise similar to that of FIG. 3A. Thereafter, the surgeon can inject a small amount of a cyanoacrylate or other bonding agent to bond together the first and second body portions. Alternatively, any of the above (and below) described energy sources can be used to “release” a bonding agent already carried within the prosthesis.

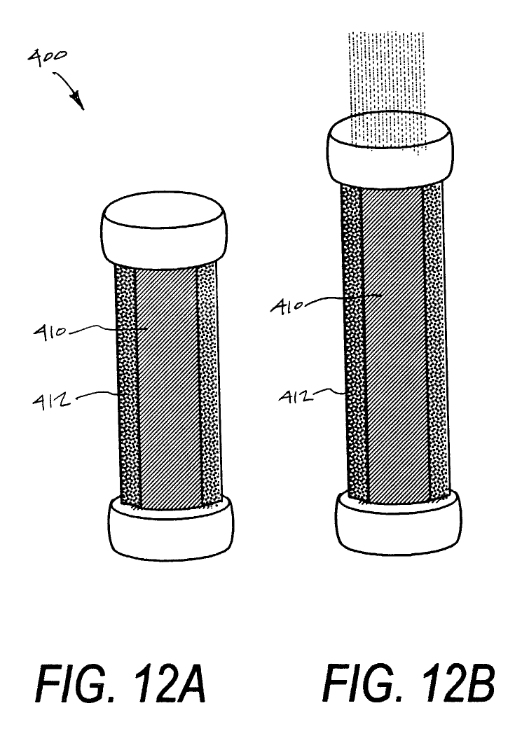

4. Type “D” prosthesis. An exemplary Type “D” prosthesis 400 in accordance with the invention is illustrated in FIG. 12, which is again a system that cooperates with a remote energy source to cause active adjustment of the overall length of the prosthesis. FIG. 12 more particularly shows a cut-away view of the prosthesis 400 that comprises an interior portion of a shape memory polymer indicated at 410.

Shape memory polymers (SMPs) demonstrate the phenomena of shape memory based on fabricating a segregated linear block co-polymer, typically of a hard segment and a soft segment. The shape memory polymer generally is characterized as defining phases that result from glass transition temperatures in the hard and a soft segments. The hard segment of SMP typically is crystalline with a defined melting point, and the soft segment is typically amorphous, with another defined transition temperature. In some embodiments, these characteristics may be reversed together with the segment's glass transition temperatures.

In one embodiment, when the SMP material is elevated in temperature above the melting point or glass transition temperature of the hard segment, the material then can be formed into a memory shape. The selected shape is memorized by cooling the SMP below the melting point or glass transition temperature of the hard segment. When the shaped SMP is cooled below the melting point or glass transition temperature of the soft segment while the shape is deformed, that temporary shape is fixed. The original shape is recovered by heating the material above the melting point or glass transition temperature of the soft segment but below the melting point or glass transition temperature of the hard segment. (Other methods for setting temporary and memory shapes are known which are described in the literature below). The recovery of the original memory shape is thus induced by an increase in temperature, and is termed the thermal shape memory effect of the polymer. The temperature can be body temperature or somewhat above or below 37° C.

The scope of the invention extends to the use of a shape memory polymer 410 that can be elevated slightly in temperature in response to energy from an external source, for example, laser light to alter its length. FIGS. 12A-12B illustrate schematically that the prosthesis can be lengthened by irradiation as the SMP portion 410 moves toward its memory position. The prosthesis is shown again with an insulative coating indicated at 412 which must be flexible.

Examples of polymers that have been utilized in hard and soft segments of SMPs include polyethers, polyacrylates, polyamides, polysiloxanes, polyurethanes, polyether amides, polyether esters, and urethanebutadiene copolymers. See, e.g., U.S. Pat. No. 5,145,935 to Hayashi; U.S. Pat. No. 5,506,300 to Ward et al.; U.S. Pat. No. 5,665,822 to Bitler et al.; and U.S. Pat. No. 6,388,043 to Langer et al, all of which are incorporated herein by reference. SMPs are also described in the literature: Ohand Gorden, Applications of Shape Memory Polyurethanes, Proceedings of the First International Conference on Shape Memory and Superelastic Technologies, SMST International Committee, pp. 115-19 (1994); Kim, et al., Polyurethanes having shape memory effect, Polymer 37(26):5781-93 (1996); Li et al., Crystallinity and morphology of segmented polyurethanes with different soft-segment length, J. Applied Polymer 62:631-38 (1996); Takahashi et al., Structure and properties of shape-memory polyurethane block copolymers, J. Applied Polymer Science 60:106-169 (1996); Tobushi H., et al., Thermomechanical properties of shape memory polymers of polyurethane series and their applications, J. Physique IV (Colloque Cl) 6:377-84 (1996)) (all of the cited literature incorporated herein by this reference).

The above described Types “A”, “B” “C” and “D” prostheses and systems represent exemplary embodiments of the invention. Other similar systems for length adjustment of an ossicular prosthesis are related and fall within the scope of the invention. For example, an external electrical energy source can be used to deliver thermal effects to a nitinol (nickel-titanium shape memory alloy) collar to provide engaging and disengaging positions for a prosthesis similar to that describe in FIGS. 3A, 3B and 4. Further, the external energy source can deliver energy to actuate either a piezoelectric or nitinol “actuator” to engage a ratchet mechanism to drive a first body relative to a second body in an implant adapted for unit-directional travel.

Further, the external energy source can be used to provide a plurality of signal frequencies that cooperate with a plurality of tuned helical coils within the implant to cause actuation of more than one piezoelectric actuator. For example, it is possible to have a first piezo-element that expands/contracts radially as a collar to grip or release a shaft. The prosthesis can carry a cooperating second piezo-element that expands/contracts axially to engage the locked collar to move the shaft axially as a manner of extending and/or locking the components of the implant. An optional third piezo-element also can act as a collar for a locking mechanism in such an active implant. Such an implant could generate substantial axial driving forces.

In a similar length-adjustable prosthesis, the external energy source can be other that an electrical source. For example, a light energy source (e.g., infrared wavelength) can be used to transmit energy through the eardrum to the prosthesis for engagement-disengagement purposes or to deliver energy to a microfluidics system. The length-adjustable prosthesis system also can use inductive heating of a fluid or interior portion of a prosthesis and thereby use such thermal energy to drive any of the length-adjustment mechanisms described above.

In a similar length-adjustable prosthesis for very slight adjustments, any external energy source can be used to apply thermal energy to at least a portion of a prosthesis body to fully polymerize a partially polymerized material to thereby alter its dimension. In one such example, a thermal “shrink-wrap” type of polymer can be used to alter the prosthesis length in various manners.

In a similar length-adjustable prosthesis adapted for very slight adjustments, an external energy source can be used to apply energy to MEMS structures known in the art (e.g., lever arms, motors, ratchets, gears, turbines, impellers and the like) to adjust the overall length of the prosthesis.

It should be further appreciated that the invention contemplates the use of self-contained voltage sources (i.e., a battery) within the prosthesis with external or internal activation mechanisms for engagement-disengagement or length-adjustment of the prosthesis, and/or external charger replenishment mechanisms.

Those skilled in the art will appreciate that the exemplary systems, combinations and descriptions are merely illustrative of the invention as a whole, and that variations of components, dimensions, and compositions described above may be made within the spirit and scope of the invention. Specific characteristics and features of the invention and its method are described in relation to some figures and not in others, and this is for convenience only. While the principles of the invention have been made clear in the exemplary descriptions and combinations, it will be obvious to those skilled in the art that modifications may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

What is claimed is:

1. A middle ear ossicular prosthesis, comprising
a prosthesis body extending about an axis between a first end surface and a second end surface;
first and second interior chambers at an interior of said prosthesis body;
a selected fluid in said first and second interior chambers; and
a flow-limiting structure intermediate said first and second interior chambers.

2. The prosthesis of claim 1 wherein the flow-limiting structure defines at least one fluid flow channel therein.

3. The prosthesis of claim 2 wherein said at least one channel has a diameter ranging from about 0.25 μm to 50 μm.

4. The prosthesis of claim 2 wherein said at least one channel has a diameter ranging from about 0.5 μm to 10 μm.

5. The prosthesis of claim 1 wherein flow-restricting structure comprises a filter that define a maximum pore dimension between about 0.25 μm and 50 μm.

6. The prosthesis of claim 1 wherein flow-restricting structure is a sintered metal filter.

7. A method of treating a middle ear disorder with an ossicular prosthesis, comprising the steps of:
providing a prosthesis assembly that defines a length between a first end and a second end thereof, wherein the assembly provides passive length adjustment means;
implanting the prosthesis between a first middle ear structure and a second middle or inner ear structure; and
causing the passive length adjustment means to adjust the length between said first and a second ends in response to forces caused by wound healing.

8. A middle ear ossicular prosthesis, comprising
a body assembly defining a length about a longtidinal axis between a first end and a second end;
the body assembly having a fluid-filled interior chamber within a medial bellows portion wherein a change of fluid volume expands or contracts the bellows portion for adjusting said length.

9. The ossicular prosthesis of claim 8 further comprising flow means for causing fluid flow into or out of said interior chamber.

10. The ossicular prosthesis of claim 9 wherein the flow means comprises a pumping mechanism.

11. The ossicular prosthesis of claim 9 wherein the flow means comprises a voltage-based pumping mechanism.

12. The ossicular prosthesis of claim 9 wherein the flow means comprises a photothermal pumping mechanism.

13. The ossicular prosthesis of claim 9 further comprising a valve mechanism between the interior chamber and a secondary fluid-filled chamber.

14. A middle ear ossicular prosthesis, comprising
a body assembly defining a length about a longtidinal axis between a first end and a second end;
the body assembly comprising first and second body portions that slidably mate about an interface thereby allowing adjustment of said length; and
an engagement mechanism for engaging the first and second body portions to thereby maintain a selected length.

15. The prosthesis of claim 14 wherein the engagement mechanism comprises a nickel titanium structure element.

16. The prosthesis of claim 14 wherein the engagement mechanism comprises a piezoelectric element.

17. The prosthesis of claim 14 further comprising an external source for coupling energy to the engagement mechanism.

18. The prosthesis of claim 17 further comprising a helical coil carried in either said first or second body portion that is responsive to a selected frequency propagated by said external source.

19. A method of treating a middle ear disorder with an ossicular prosthesis, comprising the steps of:
providing a prosthesis that defines a length between a first end and a second end thereof;
implanting the prosthesis between a first middle ear structure and a second middle or inner ear structure; and
actuating adjustment means within the prosthesis to adjust the length between the first and second ends of the prosthesis.

20. The method of claim 19 wherein the actuating step includes actuating an external energy source that cooperates with said adjustment means.

21. The method of claim 20 wherein the external energy source is selected from the class consisting of a photonic energy source, Rf energy source and magnetic energy source.

22. A middle ear ossicular prosthesis, comprising
a prosthesis body extending relative to an axis between a first end surface and a second end surface;
first and second interior chambers at an interior of said prosthesis body;
a volume of charge responsive fluid carried within said first and second interior chambers; and
a charge delivery mechanism intermediate said first and second interior chambers for causing flow of said fluid between said first and second interior chambers.

23. The prosthesis of claim 22 wherein the charge delivery mechanism comprises opposing polarity electrodes in first and second end portions of at least one channel communicating with said first and second interior chambers.

24. The prosthesis of claim 22 wherein the charge delivery mechanism further includes a voltage source for applying a voltage to said opposing polarity electrodes.

25. The prosthesis of claim 24 wherein the voltage source comprises an external Rf transmitter and a cooperating coil in said prosthesis.

26. A length-adjustable middle ear ossicular prosthesis defining first and second ends with a medial shaft portion of a shape memory polymer.

27. The prosthesis of claim 26 further comprising an insulator layer about the exterior of the shape memory polymer.
